SEQUENCE LISTING (27 sequences)

SEQ ID NO: 1 - DNA, 23 bp, Escherichia coli
  source 1..23: mol_type = genomic DNA
gagcggcaaattcttctacgcgg

SEQ ID NO: 2 - DNA, 23 bp, Escherichia coli
  source 1..23: mol_type = genomic DNA
gatgtcagaacccaattgagcgg

SEQ ID NO: 3 - DNA, 23 bp, Escherichia coli
  source 1..23: mol_type = genomic DNA
acaccatgttcgctgaactgggg

SEQ ID NO: 4 - DNA, 23 bp, Escherichia coli
  source 1..23: mol_type = genomic DNA
gaagtggtccacgttgacggcgg

SEQ ID NO: 5 - DNA, 23 bp, Escherichia coli
  source 1..23: mol_type = genomic DNA
tgaagatgtgggtaactctgcgg

SEQ ID NO: 6 - DNA, 23 bp, Escherichia coli
  source 1..23: mol_type = genomic DNA
gctgtaggagctgatgtcgtggg

SEQ ID NO: 7 - DNA, 23 bp, Escherichia coli
  source 1..23: mol_type = genomic DNA
tgctgaaaccgccgtcaacgtgg

SEQ ID NO: 8 - DNA, 23 bp, Escherichia coli
  source 1..23: mol_type = genomic DNA
agtaacggtacggcgaatcgggg

SEQ ID NO: 9 - DNA, 23 bp, Escherichia coli
  source 1..23: mol_type = genomic DNA
tggtccgatccgtactctggcgg

SEQ ID NO: 10 - DNA, 23 bp, Escherichia coli
  source 1..23: mol_type = genomic DNA
taccagaacgacaaactgaaagg

SEQ ID NO: 11 - DNA, 23 bp, Escherichia coli
  source 1..23: mol_type = genomic DNA
gaggccgccagagtacggatcgg

SEQ ID NO: 12 - DNA, 23 bp, Escherichia coli
  source 1..23: mol_type = genomic DNA
catcgcctgagcgataccgtagg

SEQ ID NO: 13 - DNA, 23 bp, Escherichia coli
  source 1..23: mol_type = genomic DNA
catcttcaaatgtaacggtacgg

SEQ ID NO: 14 - DNA, 23 bp, Escherichia coli
  source 1..23: mol_type = genomic DNA
agctgtaggagctgatgtcgtgg

SEQ ID NO: 15 - DNA, 23 bp, Escherichia coli
  source 1..23: mol_type = genomic DNA
gcacctggcgatcagctggatgg

SEQ ID NO: 16 - DNA, 23 bp, Escherichia coli
  source 1..23: mol_type = genomic DNA
aacccggattcagcatggagcgg

SEQ ID NO: 17 - DNA, 23 bp, Escherichia coli
  source 1..23: mol_type = genomic DNA
gcggaagtctttgataccggagg

SEQ ID NO: 18 - DNA, 22 bp, Escherichia coli
  source 1..22: mol_type = genomic DNA
actatccatcgcctacggtatc

SEQ ID NO: 19 - DNA, 22 bp, Escherichia coli
  source 1..22: mol_type = genomic DNA
cagcaaactatccatcgcctac

SEQ ID NO: 20 - DNA, 22 bp, Escherichia coli
  source 1..22: mol_type = genomic DNA
actatccatcgcctacggtatc

SEQ ID NO: 21 - DNA, 22 bp, Escherichia coli
  source 1..22: mol_type = genomic DNA
aactatccatcgcctacggtat

SEQ ID NO: 22 - DNA, 22 bp, Escherichia coli
  source 1..22: mol_type = genomic DNA
caggtgcaaaaccaatagagtg

SEQ ID NO: 23 - DNA, 22 bp, Escherichia coli
  source 1..22: mol_type = genomic DNA
gcgttaacatagtcaccatcca

SEQ ID NO: 24 - DNA, 22 bp, Escherichia coli
  source 1..22: mol_type = genomic DNA
ccaggtgcaaaaccaatagagt

SEQ ID NO: 25 - DNA, 275 bp, Escherichia coli
  source 1..275: mol_type = genomic DNA
tgccagcaaactatccatcgcctacggtatcgctcaggcgatgcaccgcgaaggagctgaactggcattcacctaccagaacgacaaactgaaaggccgcgtagaagaatttgccgctcaattgggttctgacatcgttctgcagtgcgatgttgcagaggatgccagcatcgacaccatgttcgctgaactggggaaagtttggccgaaatttgacggtttcgtacactctattggttttgcacctggcgatcagctggatggtgactatgtta

SEQ ID NO: 26 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 27 - RNA, 101 nt, synthetic construct
  source 1..101: mol_type = other RNA
gagcggcaaattcttctacggttttagagctagaaatagcaagttaaaataaggctagtccgttatcaaacttgaaaaagtggcaccgagtcggtgctttt

(19) United States
(12) Patent Application Publication
(10) Pub. No.: US 2025/0025562 A1
(43) Pub. Date: Jan. 23, 2025

(54) COMPOSITIONS AND METHODS FOR TREATING AN ORGANISM FOR A DISEASE CAUSED BY AN INFECTIOUS AGENT IN THE ORGANISM

(71) Applicant: Jiya Dharne, San Jose, CA (US)

(72) Inventor: Jiya Dharne, San Jose, CA (US)

(21) Appl. No.: 18/223,789

(22) Filed: Jul. 19, 2023

(51) Int. Cl. A61K 47/54

(52) U.S. Cl. CPC A61K 47/549

(57) ABSTRACT

Disclosed are compositions and methods for treating an organism for a disease caused by an infectious agent in the organism. Preferred methods comprise the steps of determining at least one protein necessary for performance of a function the infectious agent requires for survival; determining, in DNA of the infectious agent, at least one gene that can produce mRNA that can encode the protein; determining, in each gene, at least one exon arrangement that can produce the mRNA; determining at least one target sequence in the DNA, the at least one target sequence including, as to each arrangement, at least one exon in the arrangement; determining, for each target sequence, at least one protospacer adjacent motif (PAM) site, adjacent the target sequence, that can be recognized by a nuclease capable of breaking molecular bonds of the DNA; determining, for each target sequence, at least one guide RNA (gRNA) sequence complementary to the target sequence; preparing a ribonucleoprotein (RNP) complex including the gRNA sequence and the nuclease; and administering the RNP complex to the organism.

### FIELD OF THE INVENTION

The invention relates generally to compositions and methods for providing treatment, and more specifically to compositions and methods for providing treatment for diseases caused by infectious agents.

### ELECTRONIC SEQUENCE LISTING INCORPORATION BY REFERENCE

This application contains a Sequence Listing that has been submitted electronically in XML format and is hereby incorporated by reference in its entirety. The Sequence Listing is named “jd01 seqlist202401231158.xml”, was created on Jan. 26, 2024, and is 24,683 bytes in size.

### BACKGROUND OF THE INVENTION

Many antibacterial drugs, such as methicillin and penicillin, work to inhibit cell wall synthesis or metabolic pathways in pathogens. As an example, certain antibacterial drugs can be used to eliminate a pathogen by preventing it from synthesizing a specific molecule called peptidoglycan, which performs a critical function in forming cell walls of the pathogen.

However, many pathogens are naturally resistant to such drugs. The drugs are not able to pass through the cell membranes of certain pathogens. Some pathogens can modify the target sites for the drug. Other pathogens can form enzymes to disable the drugs. A number of pathogens can develop genetic mutations that allow them to resist the drugs, and through a process called conjugation, pass copies of the mutated genes to other pathogens.

To address this problem, researchers work constantly to invent new drugs to which pathogens have not developed resistance. The new drugs work for a time, but eventually, the pathogens develop resistance to them, and newer drugs must be invented and the cycle repeats. For example, methicillin resistant Staphylococcus aureus (MRSA) poses a threat because it is resistant to the drug methicillin and therefore is difficult to treat. There are very few drugs that are available to treat MRSA infections, and it is only a matter of time before MRSA becomes resistant to those as well.

Accordingly, there is a need for compositions and methods for treating organisms for diseases caused by infectious agents, that overcome the problem of drug resistance.

### SUMMARY OF THE INVENTION

The invention advantageously fills the aforementioned deficiencies by providing compositions and methods for treating an organism for a disease caused by an infectious agent in the organism.

The following descriptions of features and aspects of the invention are not meant to limit the scope of the invention, but rather to merely provide examples of preferred embodiments. Terms and phrases used are intended to have and convey their dictionary and common usage meanings, as well as or including, without limitation, the meanings specified. Terms and phrases used to convey direction or position, whether relative or absolute, are merely examples and do not limit the invention to only those directional or positional terms and phrases used, but rather the invention encompasses embodiments having components or features that are directed or positioned differently. To the extent that any refer to functionality or purpose in any way, they are intended to convey, in addition to their dictionary and common usage meanings, any arrangement, combination, or interaction of physical objects, hardware, and/or software that is suitable to any degree, whether partially or fully, for accomplishing and/or effecting the function or intended result. Further, in addition to any preferred embodiments described, the invention encompasses embodiments having features and aspects that fall into the broadest possible categories to which the described preferred features and aspects belong.

The present invention provides, in preferred embodiments, a composition and method for treating an organism for a disease caused by an infectious agent in the organism.

In an example embodiment, a new approach to treating pathogen infections against drug resistance is disclosed. The news approach does not simply inhibit genes, but rather completely knocks out genes that are required for cell survival. Therefore, unlike current drugs, the described methods and compositions work to kill pathogens before they have a chance to develop resistance. In fact, pathogens cannot develop resistance to the described methods and compositions because the pathogens cannot stop the inhibition of their genes if the genes are permanently inactivated.

In preferred embodiments, a method of treating an organism for a disease caused by an infectious agent in the organism includes one or more of the following steps/functions:
(i) determining at least one protein necessary for performance of a function the infectious agent requires for survival;
(ii) determining, in DNA of the infectious agent, at least one gene that can produce mRNA that can encode the protein;
(iii) determining, in each gene, at least one exon arrangement that can produce the mRNA;
(iv) determining at least one target sequence in the DNA, the at least one target sequence including, as to each arrangement, at least one exon in the arrangement;
(v) determining, for each target sequence, at least one protospacer adjacent motif (PAM) site, adjacent the target sequence, that can be recognized by a nuclease capable of breaking molecular bonds of the DNA;
(vi) determining, for each target sequence, at least one guide RNA (gRNA) sequence complementary to the target sequence;
(vii) preparing a ribonucleoprotein (RNP) complex including the gRNA sequence and the nuclease; and
(viii) administering the RNP complex to the organism.

The described methods can be used to develop the compositions of the present invention.

The present invention now will be described more fully hereinafter with reference to the accompanying drawings, which are intended to be viewed in conjunction with both this summary, the detailed description, and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough and complete and will fully convey the full scope of the invention to those skilled in the art.

### BRIEF DESCRIPTION OF THE DRAWINGS

FIGS. 1 and 2 illustrate use of a research tool to select a target sequence in accordance with an example embodiment of the present invention.

FIGS. 3 and 4 illustrate use of a research tool to determine a gRNA sequence in accordance with an example embodiment of the present invention.

### DETAILED DESCRIPTION OF PREFERRED EMBODIMENTS

### 1. Broad Scope and Non-Limitation of Described Embodiments

Following are more detailed descriptions of various concepts related to, and embodiments of, apparatus and methods according to the present disclosure. Various aspects of the subject matter introduced above and discussed in greater detail below may be implemented in any of numerous ways, as the subject matter is not limited to any particular manner of implementation. Examples of specific implementations and applications are provided primarily for illustrative purposes. Headings used herein are primarily for organizational purposes and do not limit the scope of the present invention.

The present invention encompasses any and all described components, elements, steps and representations, whether as or part of systems, apparatus, devices, methods, computer programs, data structures, recording mediums, and the like, and any and all combinations, permutations, and conversions between thereof. Any one or more functions that are illustrated and/or described may be implemented only in hardware, only in software, or in a combination of hardware and software, and are not limited to being implemented in only one or the other.

### 2. Overview

The present invention provides, in preferred embodiments, compositions and methods for treating an organism for diseases caused by infectious agents in the organism.

The present invention is useful for treating an organism for a disease caused by an infectious agent in the organism. Diseases that can be treated in accordance with one or more embodiments of the present invention include without limitation Staph infections, Salmonella, and strep throat. Infectious agents presently known to cause one or more of these diseases and/or others and therefore can be targeted by one or more embodiments of the present invention include without limitation Escherichia coli, Staphylococcus aureus, and Salmonella enterica.

### 2.1. Example Aspects

In an aspect, the present invention is directed to methods for treating an organism for a disease caused by an infectious agent in the organism. The methods can be carried out by one or more of people or systems, including without limitation biological systems or computer systems or an combination of biological and computer systems. Contemplated computer systems can include one or more processors, memory, and one or more programs. The one or more programs are stored in the memory and are configured to be executed by the one or more processors to treat or assist in treating a subject in need of treatment. The one or more programs include instructions for providing one or more of the steps and/or functions discussed below.

In another aspect, the present invention is directed to compositions for treating an organism for a disease caused by an infectious agent in the organism, including without limitation compositions resulting from the methods discussed herein.

### 2.2. Steps/Functions

In preferred embodiments, a method of treating an organism for a disease caused by an infectious agent in the organism includes one or more of the following steps/functions:
(i) determining at least one protein necessary for performance of a function the infectious agent requires for survival;
(ii) determining, in DNA of the infectious agent, at least one gene that can produce mRNA that can encode the protein;
(iii) determining, in each gene, at least one exon arrangement that can produce the mRNA;
(iv) determining at least one target sequence in the DNA, the at least one target sequence including, as to each arrangement, at least one exon in the arrangement;
(v) determining, for each target sequence, at least one protospacer adjacent motif (PAM) site, adjacent the target sequence, that can be recognized by a nuclease capable of breaking molecular bonds of the DNA;
(vi) determining, for each target sequence, at least one guide RNA (gRNA) sequence complementary to the target sequence;
(vii) preparing a ribonucleoprotein (RNP) complex including the gRNA sequence and the nuclease; and
(viii) administering the RNP complex to the organism.

### 2.3. Descriptions in Context of an Example Embodiment

Preferred aspects of these steps/functions will be described below, along with descriptions of an example embodiment of the present invention in which one or more are implemented.

### 3. Introduction of the Example Embodiment

In the example embodiment that will be discussed in detail herein, the present invention for treating an organism for a disease caused by an infectious agent in the organism is implemented by way of a method (and preparation of a composition) for treating an organism for a disease caused by Escherichia coli. in the organism.

The example process performs a CRISPR knockout on a selected gene. The example process is described as performed on a strain of non-pathogenic K-12 Escherichia coli, but not in a human organism. It should be understood that the process would be altered for testing and application in humans, and that such alterations are known and can be applied by those skilled in the art.

The following reference describes in detail certain procedures used in the example process: https://docs.google.com/document/d/1 Qujfi4sL_mJ6KuhbhpoGnmnul9JXcgHpchD4d4GE vKs/edit (the content of which is hereby incorporated by reference herein) (included herewith as Appendix A), except that some alterations have been made for the needs of the example process. Those alterations include instead using a different non-pathogenic K-12 Escherichia coli strain, using a different type of agar in accordance with the different Escherichia coli strain used, and using a different gRNA sequence. Regarding the agar, an LB agar (containing tryptone, yeast extract, NaCl, and agar) was used, and an LB medium broth was used to grow the strain (see https://www.atcc.org/products/47076 (the content of which is hereby incorporated by reference herein)). Furthermore, the reference describes a CRISPR knock-in process and accordingly includes a template DNA sequence for replacing the DNA sequence that is knocked-out in the reference process, but no template DNA was needed for the example process described herein because the DNA sequence knocked-out in the example process is not to be replaced in the example process.

In the example process, the fabI in the non-pathogenic K-12 Escherichia coli is targeted because it is understood to serve an important function of synthesizing cell wall lipids. Accordingly, if the fabI gene is knocked out in the Escherichia coli, the Escherichia coli will die. It should be understood, therefore, that if the process is undertaken for an organism infected with Escherichia coli, the organism can be treated by the described process to treat the infection.

As discussed above, the example process is an alternative approach to fighting drug resistant bacteria, because it does not require the use of drugs that may prove ineffective once the bacteria become resistant to those drugs. Rather than constantly making new drugs to combat infections by inhibiting genes and thus risking drug resistance, medical technicians can use CRISPR technology as described herein to permanently kill or disable pathogens because the processes can disable crucial genes rather than simply inhibiting them.

### 3.1. Determination of Protein

Reference will now be made to the step/function of determining at least one protein necessary for performance of a function the infectious agent requires for survival.

It is known that in infectious agents, certain biological functions are required for survival, such as for example, cell wall synthesis, metabolism, reproduction, and cell duplication, and that such functions are affected by proteins encoded by mRNA produced by one or more exon arrangements in one or more genes in the DNA of the infectious agent.

Accordingly, preferred embodiments include a step/function of determining at least one protein necessary for performance of a function the infectious agent requires for survival. As examples, the following proteins are known to be necessary for the following functions in the following infections agents: Protein Enoyl-Acyl is known to be necessary for cell wall lipid synthesis in Escherichia coli; Protein Transpeptidase is known to be necessary for constructing peptidoglycan for the cell wall in Staphylococcus aureus, cell division protein FtsL is known to be necessary for regulating cell division in Salmonella enterica, cell division protein ftsA is known to be necessary for regulating and anchoring the Z ring structure needed for bacterial division in Streptococcus pyogenes.

In the example process, the protein Enoyl-Acyl Carrier Protein was determined to be necessary for cell wall lipid synthesis in Escherichia coli. This determination was made by researching crucial genes in bacteria and researching the genome of Escherichia coli strain K-12 through the National Center for Biotechnology Information (NCBI) to find a gene that encodes the identified protein.

### 3.2. Determination of Gene

Reference will now be made to the step/function of determining, in DNA of the infectious agent, at least one gene that can produce mRNA that can encode the protein.

Once at least one protein necessary for performance of a function the infectious agent requires for survival has been determined, it is necessary to determine, in DNA of the infectious agent, at least one gene that can produce mRNA that can encode the protein.

Determining the at least one gene can be accomplished by any method known in the art, whether now known or developed later.

In the example process, the fabI gene was determined to produce the mRNA that encodes the Enoyl-Acyl Carrier Protein. This determination was made by analyzing the genes of Escherichia coli strain K-12 through NCBI.

### 3.3. Determination of Exon Arrangement

Reference will now be made to the step/function of determining, in each gene, at least one exon arrangement that can produce the mRNA.

Once at least one gene that can produce mRNA that can encode the protein has been determined, it is necessary to determine, in each gene, at least one exon arrangement that can produce the mRNA.

Determining the at least one exon arrangement can be accomplished by use of the CRISPR Direct Site described below (which takes into account all applicable exon arrangements when a target gene is specified) or any other method known in the art, whether now known or developed later.

In the example process, all applicable exon arrangements in the fabI gene that produce the mRNA that encodes the Enoyl-Acyl Carrier Protein were determined by specifying the fabl gene on the CRISPR Direct Site, due to the CRISPR Direct Site providing such a determination function.

### 3.4. Determination of Target Sequence

Reference will now be made to the step/function of determining at least one target sequence in the DNA, the at least one target sequence including, as to each arrangement, at least one exon in the arrangement.

Once at least one exon arrangement that can produce the mRNA has been determined, it is necessary to determine at least one target sequence in the DNA, the at least one target sequence including, as to each arrangement, at least one exon in the arrangement.

Determining the at least one target sequence can be accomplished by any method known in the art, whether now known or developed later.

Determination of the target sequence will be described below in connection with the step of determining the PAM site.

### 3.5. Determination of PAM Site

Reference will now be made to the step/function of determining, for each target sequence, at least one protospacer adjacent motif (PAM) site, adjacent the target sequence, that can be recognized by a nuclease capable of breaking molecular bonds of the DNA.

Once at least one target sequence has been determined, it is necessary to determine, for each target sequence, at least one protospacer adjacent motif (PAM) site, adjacent the target sequence, that can be recognized by a nuclease capable of breaking molecular bonds of the DNA.

Determining the at least one PAM site can be accomplished by any method known in the art, whether now known or developed later.

In the example process, reference is made to a research tool located at https://chopchop.cbu.uib.no (the content of which is hereby incorporated by reference herein) (referred to herein as “ChopChop Site”). This tool provides a user interface for searching and organizing genetic research results. Using this tool, it is possible to determine a target sequence and adjacent PAM site in the gene. The PAM site is a short DNA sequence that can be targeted by the Cas9 nuclease for cleavage.

Preferably, the first presented option with the highest target efficiency is selected, however, it is important to take into consideration if alternative splicing would result in the same product the gene produced. Alternative splicing is a cellular mechanism that allows the pre-mRNA (RNA containing sequences that code for certain proteins), to modify the arrangement of protein coding sequences (exons). This process produces a diversity of mRNAs from a single gene because the pattern of exons can be rearranged. (For example, if a certain sequence that was used as exons is knocked out with CRISPR, alternative splicing may result in the usage of other sequences to still result in the same protein).

As an example, the following reference describes a process for selecting a target sequence and adjacent PAM site: https://currentprotocols.onlinelibrary.wiley.com/doi/full/10.1002/cpz1.46 (the content of which is hereby incorporated by reference herein) (included herewith as Appendix B).

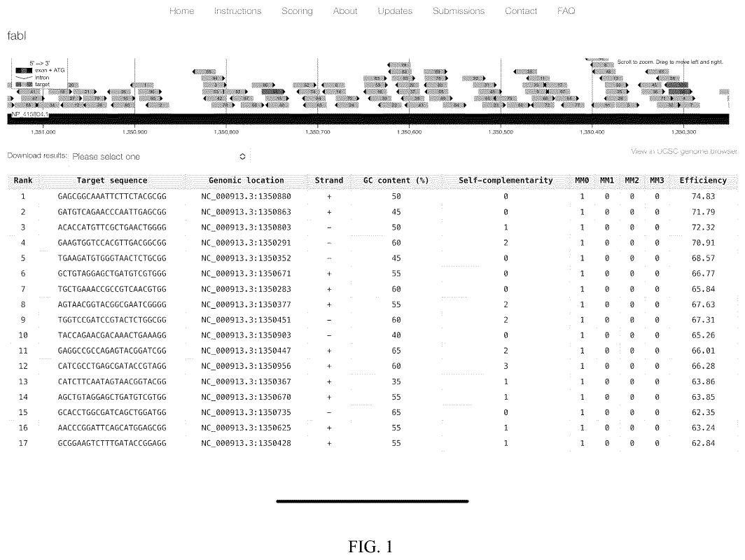

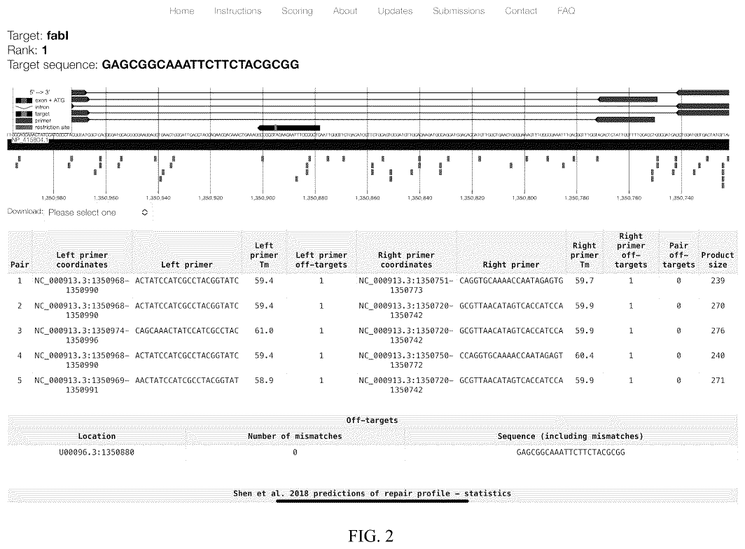

FIGS. 1 and 2 illustrate use of the ChopChop Site to select a target sequence, with adjacent PAM site, that includes at least one exon in an exon arrangement in the fabI gene that can produce mRNA that can encode the protein determined to be necessary for cell wall synthesis in Escherichia coli.

All target site options for the gene will be listed, therefore clicking on an individual target site will show the rank, gRNA target sequence, and gene on the top left of the page, a visualization of primers, the target site within the genome, restriction enzymes, possible off-targets, and repair predictions.

### 3.6. Determination of gRNA

Reference will now be made to the step/function of determining, for each target sequence, at least one guide RNA (gRNA) sequence complementary to the target sequence.

Once at least one PAM site has been determined, it is necessary to determine, for each target sequence, at least one guide RNA (gRNA) sequence complementary to the target sequence.

Determining the at least one guide RNA (gRNA) sequence can be accomplished by any method known in the art, whether now known or developed later.

In the example process, after the target sequence is determined, the gRNA (containing a sequence complementary to the determined target sequence) that will bind to the target sequence in the fabI gene for a knockout can be determined using a research tool located at https://crispr.dbcls.jp/(the content of which is hereby incorporated by reference herein) (referred to herein as “CRISPR Direct Site”). This tool provides a user interface for determining the gRNA. Using this tool, it is possible to input the target sequence and adjacent PAM sequence, and then select the species of organism that is being targeted (in this example, Escherichia coli.). The tool will then generate the complementary gRNA sequence.

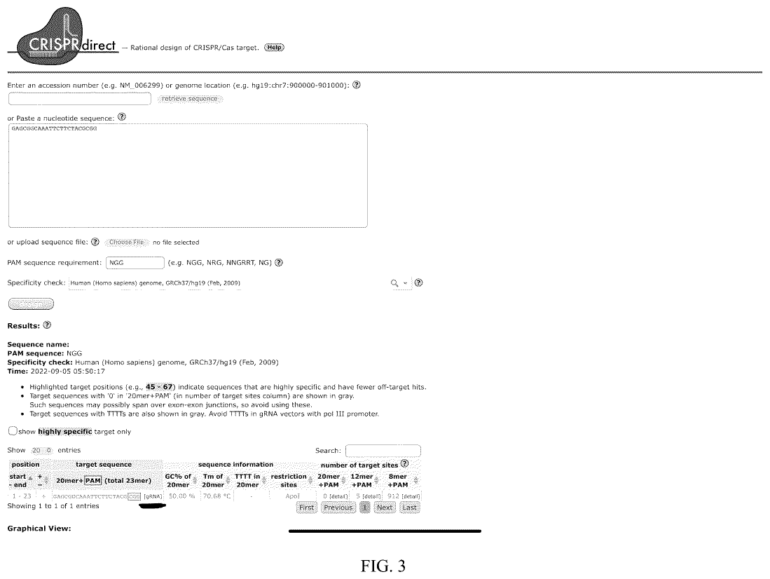

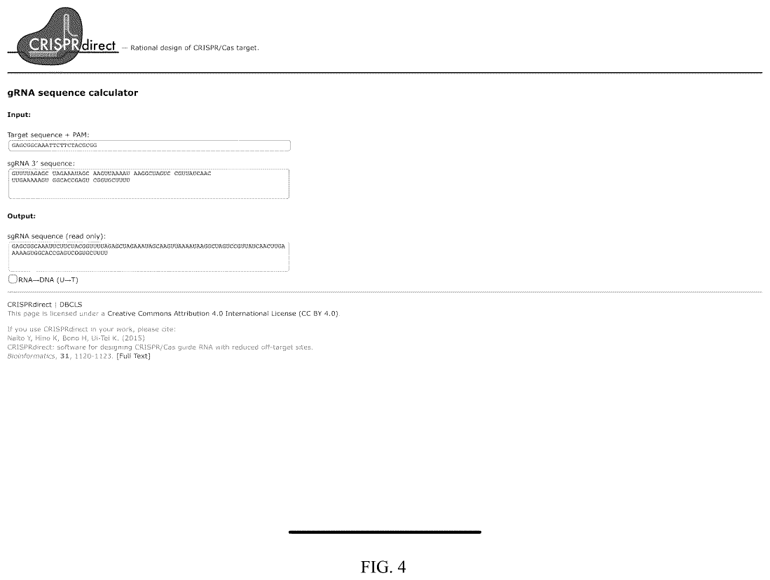

FIGS. 3 and 4 illustrate use of the CRISPR Direct Site to determine a gRNA sequence complementary to the determined target sequence, with adjacent PAM site, that includes at least one exon in an exon arrangement in the fabI gene that can produce mRNA that can encode the protein determined to be necessary for cell wall synthesis in Escherichia coli.

The CRISPR Direct Site enables a user to identify a gene that is to be targeted, and the type of CRISPR mechanism that is to be used (e.g., CRISPR knock-out). Based on these identifications, the CRISPR Direct Site provides a list of all gRNAs that can be used, and one or more can be chosen after alternative splicing and off-target effects are considered, as discussed above.

The target sequence in the top left corner of FIG. 4 is the gRNA sequence+PAM site sequence, however the gRNA sequence when ordered should not include the PAM site sequence, and therefore the gRNA sequence is modified through the CRISPR Direct Site. The actual target site that the gRNA will bind to in the gene is provided in the CHOPCHOP Site under the black arrow of the page that provides the target sequence.

### 3.7. Preparing RNP Complex

Reference will now be made to the step/function of preparing a ribonucleoprotein (RNP) complex including the gRNA sequence and the nuclease.

Once at least one guide RNA (gRNA) sequence has been determined, it is necessary to prepare a ribonucleoprotein (RNP) complex including the gRNA sequence and the nuclease.

Preparing the ribonucleoprotein (RNP) complex can be accomplished by any method known in the art, whether now known or developed later.

In the example process, once the gRNA sequence has been determined, the RNP complex including the gRNA sequence and the nuclease is prepared. In the example process, the Cas9 nuclease is being used. The Cas9 protein can be obtained by known techniques and further is widely available for commercial purchase. The Cas9 protein anneals with the gRNA sequence to form the RNP complex. The RNP complex can then be prepared by annealing the Cas9 protein and gRNA sequence together by incubating the mixture first in the fridge then in a warm water bath. 3.8. Administering RNP Complex

Reference will now be made to the step/function of administering the RNP complex to the organism.

Once the RNP complex has been prepared, it can be administered to the organism.

Administration of the RNP complex to the organism can be accomplished by known uses of bacteriophages or any other method known in the art, whether now known or developed later.

In the example process, the RNP complex containing the gRNA and the Cas9 nuclease is injected into the organism that is infected by the Escherichia coli. For testing and application in humans, the example process would be altered. Such alterations are known and can be applied by those skilled in the art.

### 4. Broad Scope

Accordingly, while the present invention may be described in terms of specific embodiments, the present invention is not limited to these disclosed embodiments. Upon reading this disclosure, many modifications and other embodiments of the present invention will come to mind of those skilled in the art to which this invention pertains, and those are intended to be and are covered by this disclosure and the appended claims. The scope of the present invention should be determined by proper interpretation and construction of the appended claims and their legal equivalents, as understood by those of skill in the art relying upon the disclosure in this specification and the attached drawings.

What is claimed is:

1. A method of treating an organism for a disease caused by an infectious agent in the organism, comprising the steps of:
determining at least one protein necessary for performance of a function the infectious agent requires for survival;
determining, in DNA of the infectious agent, at least one gene that can produce mRNA that can encode the protein;
determining, in each gene, at least one exon arrangement that can produce the mRNA;
determining at least one target sequence in the DNA, the at least one target sequence including, as to each arrangement, at least one exon in the arrangement;
determining, for each target sequence, at least one protospacer adjacent motif (PAM) site, adjacent the target sequence, that can be recognized by a nuclease capable of breaking molecular bonds of the DNA;
determining, for each target sequence, at least one guide RNA (gRNA) sequence complementary to the target sequence;
preparing a ribonucleoprotein (RNP) complex including the gRNA sequence and the nuclease; and
administering the RNP complex to the organism.

2. The method of claim 1, wherein the nuclease is Cas9.

3. The method of claim 1, wherein the infectious agent is selected from the group consisting of methicillin resistant Staphylococcus aureus (MRSA), Escherichia coli, Staphylococcus aureus, Salmonella enterica, and Streptococcus pyogenes.

4. The method of claim 1, wherein the function materially contributes to one or more of the following cellular processes: cell wall synthesis, cell membrane synthesis, metabolism, reproduction, and cell duplication.

5. The method of claim 4, wherein the function materially contributes to synthesis of cell wall lipids.

6. A pharmaceutical composition useful for treating an organism for a disease caused by an infectious agent in the organism, comprising a ribonucleoprotein (RNP) complex including a guide RNA (gRNA) sequence and a nuclease; wherein
determination has been made as to:
at least one protein necessary for performance of a function the infectious agent requires for survival;
in DNA of the infectious agent, at least one gene that can produce mRNA that can encode the protein;
in each gene, at least one exon arrangement that can produce the mRNA;
at least one target sequence in the DNA, the at least one target sequence including, as to each arrangement, at least one exon in the arrangement; and
for the at least one target sequence, at least one protospacer adjacent motif (PAM) site adjacent the at least one target sequence; and wherein
the gRNA sequence is complementary to the at least one target sequence;
the PAM site can be recognized by the nuclease; and
the nuclease is capable of breaking molecular bonds of the DNA.

7. The method of claim 6, wherein the nuclease is Cas9.

8. The method of claim 6, wherein the infectious agent is selected from the group consisting of methicillin resistant Staphylococcus aureus (MRSA), Escherichia coli, Staphylococcus aureus, Salmonella enterica, and Streptococcus pyogenes.

9. The method of claim 6, wherein the function materially contributes to one or more of the following cellular processes: cell wall synthesis, cell membrane synthesis, metabolism, reproduction, and cell duplication.

10. The method of claim 9, wherein the function materially contributes to synthesis of cell wall lipids.
